(19) United States
(12) Patent Application Publication
(10) Pub. No.: US 2004/0180961 A1
(43) Pub. Date: Sep. 16, 2004

(54) Compositions and preparation methods for bioavailable oral aceclofenac dosage forms

(76) Inventors: Beom-Jin Lee, Seoul (KR); Dong-Won Lee, Seoul (KR); Tae-Wan Kim, Kangwon-do (KR)

Correspondence Address: MUSERLIAN AND LUCAS AND MERCANTI, LLP, 475 PARK AVENUE SOUTH, NEW YORK, NY, 10016, US

(21) Appl. No.: 10/481,972

(22) PCT Filed: Jun. 25, 2002
(86) PCT No.: PCT/KR02/01210

(30) Foreign Application Priority Data
Jul. 3, 2001 (KR) 2001-0039387

(51) Int. Cl. A61K 31/195; A61K 9/20; A61K 9/14

(52) U.S. Cl. 514/567; 424/486

(57) ABSTRACT

There are provided compositions and preparation methods for bioavailable oral aceclofenac dosage forms. More particularly, the compositions containing water-insoluble aceclofenac, a polymeric base selected from the group consisting of polyvinyl, methyl cellulose, ethyl cellulose, hydroxypropylmethylcellulose, carboxy methyl cellulose, glyceryl monostearate, carbamer, and poloxamer, and surfactant. The compositions of the present invention can be formulated into compressed particles, granules, tablets, capsules or even semisolid preparations, which significantly increase the bioavilablity due to the improvement of dissolution of the drug in gastrointestinal tract, and reduce the manufacturing cost by simple process.

### TECHNICAL FIELD

The present invention relates to a pharmaceutical preparation containing aceclofenac for oral administration, and more particularly, to an oral preparation comprising poorly water-soluble aceclofenac, a polymeric base selected from the group consisting of polyvinylpyrrolidone, methylcellulose, ethylcellulose, hydroxypropylmethylcellulose, carboxymethylcellulose, glycerolmonostearate, carbamer and poloxamer, and a surfactant. Also, the present invention is concerned with a method of preparing such an oral preparation.

### BACKGROUND ART

Aceclofenac, represented by the following Formula 1, is a non-steroidal anti-inflammatory drug (NSAID), and its preparation method and efficacy are disclosed in detail in publications including Pat. No. WO 99/62865 and WO 99/55660, and the journal “Drugs” (Vol. 52(1), 113-124 (1996))

Formula 1

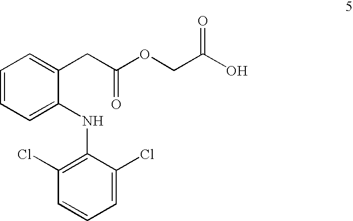

However, when being administered to the human body, a poorly water-soluble aceclofenac has a low solubility and dissolution rate in digestive fluid and its absorption is delayed, thereby lowering its bioavailabilty. Thus, there have been various attempts to enhance solubility and dissolution rate of poorly water-soluble drugs containing aceclofenac.

Pat. No. WO 90/06746 discloses a dehydrated oil-in-distilled water emulsion prepared by dehydrating an oil-in-distilled water emulsion comprising a fat-soluble drug, an emulsifier, water-soluble carbohydrate and distilled water. However, this patent is concerned about a fat-soluble drug, does not describe whether aceclofenac is applied to this method or not. In addition, because of containing distilled water in an amount of 20-29 wt %, thus not achieving complete dehydration, the dehydrated oil-in-distilled water emulsion composition is problematic in stability during storage.

JP H08-157362A discloses a fat-soluble material-containing powder preparation, which is prepared by emulsifying a fat-soluble compound in an aqueous solution containing a water-soluble polymeric substance and then spraying the resulting emulsion solution. However, in this method, excessive distilled water is used and there is no mention of its application to aceclofenac.

Pat. No. WO 00/00179, which is disclosed by the present inventors, provides a solid dispersed preparation for poorly distilled water-soluble drugs, which is prepared by dissolving or dispersing the poorly water-soluble drugs in an oil, a fatty acid or a mixture thereof, mixing the solution or dispersion in a water-soluble polymeric base, and drying the mixture. Herein, disclosed are various poorly water-soluble drugs including aceclofenac and lovastatin, and polyethylene glycol (PEG) and polyvinylpyrrolidone (PVP) as examples of the water-soluble polymeric base. In an example of this invention, there is provided a solid dispersed preparation containing aceclofenac, using only PEG among the water-soluble polymeric base. Its solubility and dissolution does not improve definitely in the solid dispersed preparation containing aceclofenac. Moreover, there is no mention of efficacy and stability of a pharmaceutical preparation prepared by using PVP.

Leading to the present invention, the intensive and thorough research into various compositions capable of improving dissolution of aceclofenac and thus increasing its bioavailabilty, conducted by the present inventors, resulted in the finding that dissolution ability and bioavailabilty of aceclofenac is improved, when a new pharmaceutical preparation for oral administration is comprised of poorly water-soluble aceclofenac, a polymeric base selected from the group consisting of polyvinylpyrrolidone, methylcellulose, ethylcellulose, hydroxypropylmethylcellulose, carboxymethylcellulose, glycerolmonostearate, carbamer, and poloxamer, and surfactant. Moreover it is disclosed that a solid powder preparation prepared by using PVP among the polymeric bases, pharmaceutically further processed solid preparations such as compressed particles, granules, tablets or capsules, and semisolid preparations capable of being filled into soft capsules is effective of the pharmaceutical preparation of the present invention.

### DISCLOSURE OF THE INVENTION

It is therefore an object of the present invention to provide a pharmaceutical preparation containing aceclofenac for oral administration, which improves dissolution of aceclofenac in the gastrointestinal tract and increases its bioavailabilty, thus diminishes the adiministrated amount.

It is another object of the present invention to provide a pharmaceutical preparation for oral administration, comprising aceclofenac, a polymeric base, and a surfactant.

It is a further object of the present invention to provide a pharmaceutical preparation containing aceclofenac for oral administration, increasing its dissolution rate and bioavailabilty in the gastrointestinal tract through use of PVP as a polymeric base for aceclofenac.

It is a still further object of the present invention to provide a method of preparing such a pharmaceutical preparation.

### BRIEF DESCRIPTION OF THE DRAWINGS

FIG. 1 is a graph in which concentration of aceclofenac in blood is plotted against time after oral administration of a capsule preparation containing 70 mg of aceclofenac according to the present invention and a commercially available preparation (Airtal, Daewoong Pharmaceutical Co. Ltd, Korea) containing 100 mg of aceclofenac.

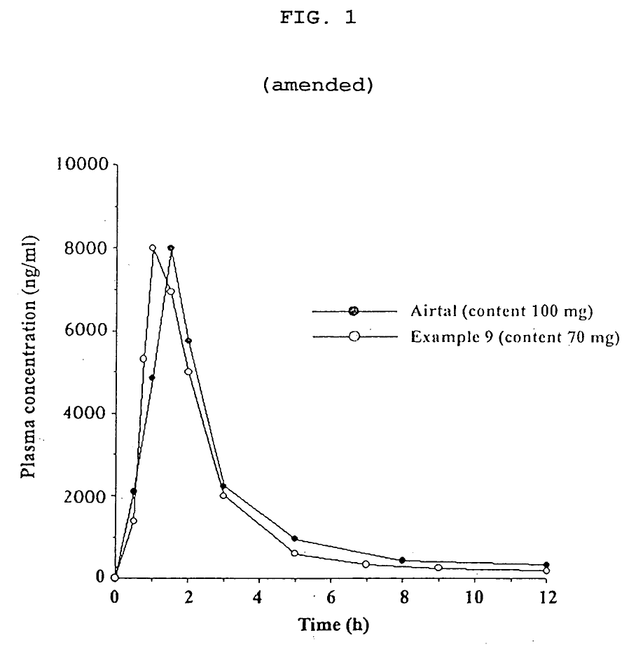

### BEST MODE FOR CARRYING OUT THE INVENTION

To achieve the above objects, in accordance with the present invention, there is provided a pharmaceutical preparation for oral administration, comprising poorly water-soluble aceclofenac, a polymeric base selected from the group consisting of polyvinylpyrrolidone, methylcellulose, ethylcellulose, hydroxypropylmethylcellulose, carboxymethylcellulose, glycerolmonostearate, carbamer, and poloxamer, and a surfactant. More particularly, the present invention is related to solid preparations for oral administration, which is prepared by spray-drying or coprecipitating (dissolve-drying) solution containing aceclofenac, a polymeric base selected from the group consisting of polyvinylpyrrolidone, methylcellulose, ethylcellulose, hydroxypropylmethylcellulose, carboxymethylcellulose, glycerolmonostearate, carbamer, and poloxamer, and a surfactant. Also, the present invention is related to a viscous semisolid preparations prepared by simply mixing and then milling the mixture comprising aceclofenac, a polymeric base selected from the group consisting of polyvinylpyrrolidone, methylcellulose, ethylcellulose, hydroxypropylmethylcellulose, carboxymethylcellulose, glycerolmonostearate, carbamer, and poloxamer, and a surfactant.

The polymeric base useful for pharmaceutical preparation of the present invention is selected from the group consisting of polyvinylpyrrolidone, methylcellulose, ethylcellulose, hydroxypropylmethylcellulose, carboxymethylcellulose, glycerolmonostearate, carbamer, and poloxamer, and most preferably, polyvinylpyrrolidone (PVP). PVP, which is also called kollidon, plasdone, or povidone, is a synthetic polymer composed of linear 1-vinyl-2-pyrrolidinone, having various average molecular weights ranging from 2,500 to 3,000,000. PVP is generally used as a pharmaceutical additive, functioning as a binder, a coating base, a dissolution-increasing base, a disintegrant or an emulsifier, and especially, serves to disperse or stabilize drugs or increase their viscosity in semi-solid preparations. PVP is water-insoluble and does not have a definite melting point, but it is soft at temperatures over 150° C. Such a poorly water-soluble drug is typically formulated into a solid dispersed preparation prepared by dissolving the drug in an organic solvent and then dispersing, thus increasing its solubility and dissolution rate.

The surfactant useful for the pharmaceutical preparation of the present invention, which has polar and non-polar groups, has a hydrophilic, lipophilic or hydrophilic-lipophilic balanced nature. Hydrophilic-lipophilic balanced nature is represented to HLB, which is used as a principle criterion in selection of various surfactants. The surfactant functions to lower surface tension between distilled water and oil, and thus is typically used as an emulsifier, a surface adsorbent, a wetting agent and a dispersing agent, as well as an auxiliary dissolving agent when being present at a concentration higher than critical micelle concentration (CMC), thereby being wildly utilized in increasing solubility and bioavailabilty of poorly water-soluble drugs. Examples of the surfactant useful in the present invention include, but are not limited to, sodium lauryl sulfate and its derivatives, poloxamer and its derivatives, medium chain triglyceride (MCT), labrasol, transcutol, labrafil, labrafac, polysorbates, which are exemplified as polyoxyethylene sorbitan monolaurate (Tween 20), polyoxyethylene sorbitan monopalmitate (Tween 40) and polyoxyethylene sorbitan monostearate (Tween 60), polyoxyethylene sorbitan monooleate (Tween 80), sorbitan esters, which are exemplified as sorbitan monolaurate (Span 20), sorbitan monopalmitate (Span 40), sorbitan monostearate (Span 60), sorbitan monooleate (Span 80), sorbitan trilaurate (Span 25), sorbitan trioleate (Span 85) and sorbitan tristearate (Span 65), cremophor, PEG-60 hydrogenated castor oil, PEG-40 hydrogenated castor oil, sodium lauryl glutamate, and disodium cocoamphodiacetate. Preferred surfactants are anionic surfactants, such as sodium lauryl sulfate and its derivatives, nonionic surfactants, such as Tween 20, 40, 60 or 80, and sorbitan esters, such as Span 20, 40, 60, 80, 25, 85 or 65, and sodium lauryl sulfate and Tween 80 are most preferable.

The pharmaceutical preparation for oral administration according to the present invention further includes a water-soluble polymeric bases, which are exemplified as gelatin, gums, carbohydrates, cellulose and its derivatives, polyethylene oxide and its derivatives, polyvinyl alcohol, polyacrylic acid and its derivatives such as carbamer, poloxamer, polymethylacrylate, and inorganic compounds. Also, the pharmaceutical preparation for for oral administration according to the present invention further includes other polymeric bases capable of forming semi-solid preparation such as typical ointments or suppositories, which are exemplified as glycerylmonostearate, cacao butter, laurin, whitepsol and a hydrophilic ointment base or absorbant ointment base, and the water-soluble polymeric bases which are exemplified as gelatin, gums, carbohydrates, cellulose and its derivatives such as sodium carboxymethyl cellulose or hydroxypromethyl cellulose, polyethylene oxide and its derivates, polyvinyl alcohol, polyacrylic acid and its derivatives such as carbamer, polymethacrylates and poloxamer. The polymeric bases are contained in semi-solid preparations.

In addition, the pharmaceutical preparation for oral administration according to the present invention further includes various substances, which is used pharmaceutically, within an amount not negatively affecting efficacy thereof. Examples of the substances useful in the present invention include fatty acid or fatty acid alcohol which enhances a dissolution and bioavailabilty of aceclofenac by increasing its solubility and absorption in the gastrointestinal through dispersion and emulsion with distilled water upon being orally administered, oil which assists dissolution or dispersion of aceclofenac and is used as an emulsifier, an antioxidant which prevents oxidation of the oral preparation, a disintegrant which assists faster release of drugs, a lubricant which improves molding grade, and a foaming agent which enhances foaming of pharmaceutical preparations.

Examples of fatty acid or fatty acid alcohol useful in the pharmaceutical preparation according to the present invention include, but are not limited to, oleic acid, stearyl alcohol, myristic acid, linoleic acid or lauric acid, capric acid, caprylic acid, and caproic acid. Oleic acid is more preferable.

Examples of oil useful in the pharmaceutical preparation according to the present invention include, but are not limited to, captylic/capric triglyceride, α-bisabolol, tocopheryl acetate, liposome, phospholipid such as phosphatidylcholine, di-c12-13 alkyl malate, cococaptylate/caprate, cetyl octanoate, hydrogenated castor oil, and other pharmaceutically acceptable oils.

Examples of the antioxidant useful in the pharmaceutical preparation according to the present invention include, but are not limited to, butylated hydroxytoluene (BHT), sodium bisulfite, α-tocorpherol, vitamin C, β-carotin, ascorbylpamitate, tocopherol acetate, fumaric acid, nalic acid, butylated hydroxyanisole, propyl gallate, and sodium ascorbate. Such antioxidants are contained in an amount of 0.0001-10% to the amount of the pharmaceutical preparation.

Examples of the disintegrant useful in the pharmaceutical preparation according to the present invention include, but are not limited to, croscarmellose sodium, sodium starch glycolate (Primojel), microcrystalline cellulose (Avicel), crospovidone (Polyplasdone), other commercially available PVP, low-substituted hydroxypropylcellulose, alginic acid, calcium salts and sodium salts of carboxy methyl cellulose (CMC), colloidal silicon dioxide, guar gum, magnesium aluminum silicate, methylcellulose, powdered cellulose, starch, and sodium alginate. The disintegrant, added to solid powder preparation for oral administration, can be added in formulating preparations such as compressed particles, granules, tablets and capsules.

Examples of the lubricant, which improves reformation of preparation, useful in the pharmaceutical preparation according to the present invention include, but are not limited to, magnesium stearate or amorphous fumed silica (Cab-O-sil), talc, and other pharmaceutically used lubricants.

Examples of the foaming agent useful in the pharmaceutical preparation according to the present invention include, but are not limited to, NaHCO₃ and Na₂CO₃.

Composition of aceclofenac, the polymeric base (PVP as a representative example) and the surfactant contained in the pharmaceutical preparation according to the present invention is controlled by its preparation method. For example, when being prepared using the spray-drying or coprecipitation, the oral preparation contains aceclofenac in an amount of 56-84 parts by weight, the polymeric base in an amount of 112-168 parts by weight, and the surfactant in an amount of 56-168 parts by weight. More preferably, especially when being prepared using the dissolve-drying, the pharmaceutical composition contains aceclofenac in amounts of 56-84 parts by weight, the polymeric base in an amount of 112-168 parts by weight, the polymeric base in an amount of 112-308 parts by weight, sodium lauryl sulfate in an amount of 56 to 84 parts by weight, Tween 80 in an amount of 15 to 20 parts by weight, fatty acid or fatty acid alcohol (preferably, oleic acid) in an amount of 15 to 20 parts by weight, and the antioxidant (preferably, butylated hydroxytoluene) in an amount of 0.15 to 0.20 parts by weigh.

When being orally administered, the pharmaceutical preparation for oral administration according to the present invention has increased its bioavailabilty 2-4 times higher than a commercially available preparation (Airtal, Daewoong Pharmaceutical Co. Ltd, Korea). That is, the pharmaceutical preparation, which contains small amount (30-80 mg) of aceclofenac, has efficacy corresponding to that of the conventional preparation (Airtal) containing 100 mg of aceclofenac. Content of aceclofenac contained in the pharmaceutical preparation for oral administration may be selected in a proper amount, taking consideration of economy and stability, preferably 30-150 mg (once per day, for controlled release), preferably 30-80 mg, and even more preferably 40-70 mg.

When being prepared using the spray-drying or coprecipitation, the pharmaceutical preparation according to the present invention has excellent dissolution ability and bioavailabilty, which is achieved by dissolving aceclofenac, the polymeric base (for example, PVP) and the surfactant in a hydrophilic solution or a mixture of a hydrophilic solvent and distilled water, and then spray-drying or dissolve-drying the resulting solution. The term “hydrophilic solvent”, as used herein, refers to a solvent mixed with distilled water, and the term “hydrophilic solution” refers to the solution prepared by dissolving solute in hydrophilic solvent. The hydrophilic solvent is exemplified as acetone, ethanol, tetrahydrofuran, propanol and butanol, but is not limited to these and should be suitably selected, considering ability to dissolve solutes and toxicity to human body. Preferably, hydrophilic solvent is a mixture of acetone, ethanol and distilled water in a volume ratio of 0.52:0.52:0.251, and more preferably, 1:1:0.5. The mixture of acetone, ethanol and distilled water is used in an amount of 10-100 ml per 1 g drug, preferably, 1,530 ml upon using the spray-drying, and 26 ml upon using the coprecipitation, and in the present invention, for convenience, 25 ml and 5 ml upon using the spray-drying and coprecipitation, respectively.

The solution containing aceclofenac, PVP and the surfactant can be formulated into solid power by performing a drying process common in the art. For example, solid powder can be acquired by using a spray drier and a fluidized-sprayer, which is common in the art, in spray-drying, or drying naturally after vaporizing or heating to about 50° C. in coprecipitation. The resultant solid powder may be ground and then formulated into compressed particles, pellets, granules or tablets, or mixed with the lubricant and then filled into capsules.

In addition, the pharmaceutical preparation according to the present invention can be prepared by melted-mixing or simple-mixing. In this case, a viscous semi-solid preparation contains aceclofenac in an amount of 56-84 parts by weight, a polymeric base (for example, PVP) in an amount of 10-168 parts by weight, and a surfactant in an amount of 56-168 parts by weight. Preferably, the pharmaceutical preparation according to the present invention contains aceclofenac in an amount of 56-84 parts by weight, the polymeric base (for example, PVP) in an amount of 10-168 parts by weight, sodium lauryl sulfate in an amount of 56-84 parts by weight, Tween 80 in an amount of 50-160 parts by weight, fatty acid or fatty acid alcohol (preferably, oleic acid) in an amount of 50-160 parts by weight, and the antioxidant (preferably, butylated hydroxytoluene) in an amount of 1.5-2.0 parts by weight.

Preparation of the viscous semi-solid preparation containing aceclofenac, a polymeric base (for example, PVP) and a surfactant may be accomplished through the typical method of preparing ointments or suppositories, self-microemulsion or self-emulsion, and the resultant viscous semi-solid preparation can be filled into the soft capsule.

The pharmaceutical preparation according to the present invention may be provided into various forms including compressed particles, granules, tablets, and capsules. For example, the solid powder obtained by using the spray-drying or coprecipitation can be formulated into compressed particles, granules, tablets, and capsules, using a pharmaceutical method well known to those of ordinary skill in the art. For example, the solid powder preparation obtained by spray-drying or coprecipitation may be mixed with the lubricant and the disintegrant, which are pharmaceutically acceptable, and then tableted to give a tablet. Also, the solid power preparation obtained by spray-drying or coprecipitation may be filled into capsules after being mixed with the lubricant. Also a viscous semisolid preparation may be filled into capsules, preferably, soft capsules.

In accordance with an example of the present invention, the pharmaceutical preparation for oral administration according to the present invention, containing aceclofenac, the polymeric base (for example, PVP), the surfactant (for example, sodium lauryl sulfate or Tween 80), and fatty acid or fatty acid alcohol (for example, oleic acid) shows improvements in its dissolution rate and bioavailabilty in artificial gastric juice, which are higher than that of aceclofenac powder and the commercially available preparation. In addition, the pharmaceutical preparation has significantly improved its dissolution rate and, when being administered in rat and human. Moreover, upon including additionally fatty acid or fatty acid alcohol, oil, the antioxidant, the disintegrant, and/or the foaming agent, the pharmaceutical preparation shows a greater increase in its dissolution rate and bioavailabilty.

The present invention will be explained in more detail with reference to the following examples. However, the following examples are provided only to illustrate the present invention, and the present invention is not limited to them. Therefore, it will be apparent to one skilled in the art that various changes and modifications can be made therein without departing from the spirit and scope thereof.

### EXAMPLE 1

1 g of aceclofenac was dissolved in 20 ml of a mixture of acetone and ethanol in a volume ratio of 1:1, and 1 g of PVP was then added thereto and completely dissolved, giving a clear solution. 0.2 g of sodium lauryl sulfate was added to the solution, together with 5 ml distilled water, and completely dissolved until being transparent. The resultant reaction mixture was sprayed and then dried to produce a solid powder preparation using a procedure as will be described in Experimental Example 1, below.

### EXAMPLE 2

1 g of aceclofenac was dissolved in 20 ml of a mixture of acetone and ethanol in a volume ratio of 1:1, and 1 g of PVP was then added thereto and completely dissolved, giving a transparent solution. 2 g of sodium lauryl sulfate was added to the solution, together with 5 ml distilled water, and completely dissolved to give a clear solution. The resultant mixture was sprayed and then dried to produce a solid powder preparation using a procedure as will be described in Experimental Example 1, below.

### EXAMPLE 3

1 g of aceclofenac was dissolved in 20 ml of a mixture of acetone and ethanol in a volume ratio of 1:1, and 1 g of PVP was then added thereto and completely dissolved, giving a clear solution. 4 g of sodium lauryl sulfate was added to the solution, together with 5 ml distilled water, and completely dissolved to give a clear solution. The resultant mixture was sprayed and then dried to produce a solid powder preparation using a procedure as will be described in Experimental Example 1, below.

### EXAMPLE 4

1 g of aceclofenac and 0.25 g of Tween 80 were dissolved in 20 ml of a mixture of acetone and ethanol in a volume ratio of 1:1, and 1 g of PVP was then added thereto and completely dissolved, giving a clear solution. 2 g of sodium lauryl sulfate was added to the solution, together with 5 ml distilled water, and completely dissolved to give a clear solution. The resultant reaction mixture was sprayed and then dried to produce a solid powder preparation using a procedure as will be described in Experimental Example 1, below.

### EXAMPLE 5

1.5 g of aceclofenac and 0.25 g of Tween 80 were dissolved in 20 ml of a mixture of acetone and ethanol in a volume ratio of 1:1, and 1 g of PVP was then added thereto and completely dissolved, giving a clear solution. 2 g of sodium lauryl sulfate was added to the solution, together with 5 ml distilled water, and completely dissolved to give a clear solution. The resultant reaction mixture was sprayed and then dried to produce a solid powder preparation using a procedure as will be described in Experimental Example 1, below.

### EXAMPLE 6

2 g of aceclofenac and 0.25 g of Tween 80 were dissolved in 20 ml of a mixture of acetone and ethanol in a volume ratio of 1:1, and 1 g of PVP was then added thereto and completely dissolved, giving a clear solution. 2 g of sodium lauryl sulfate was added to the solution, together with 5 ml distilled water, and completely dissolved to give a clear solution. The resultant reaction mixture was sprayed and then dried to produce a solid powder preparation using a procedure as will be described in Experimental Example 1, below.

### EXAMPLE 7

1 g of aceclofenac and 0.25 g of Tween 80 were dissolved in 20 ml of a mixture of acetone and ethanol in a volume ratio of 1:1, and 1 g of PVP was then added thereto and completely dissolved, giving a clear solution. 1 g of sodium lauryl sulfate was added to the solution, together with 5 ml distilled water, and completely dissolved to give a clear solution. The resultant reaction mixture was sprayed and then dried to produce a solid powder preparation using a procedure as will be described in Experimental Example 1, below.

### EXAMPLE 8

1 g of aceclofenac and 0.25 g of Tween 80 were dissolved in 20 ml of a mixture of acetone and ethanol in a volume ratio of 1:1, and 2 g of PVP was then added thereto and completely dissolved, giving a clear solution. 1 g of sodium lauryl sulfate was added to the solution, together with 5 ml distilled water, and completely dissolved to give a clear solution. The resultant reaction mixture was sprayed and then dried to produce a solid powder preparation using a procedure as will be described in Experimental Example 1, below.

### EXAMPLE 9

1 g of aceclofenac, 0.25 g of Tween 80 and 0.25 g of oleic acid were dissolved in 20 ml of a mixture of acetone and ethanol in a volume ratio of 1:1, and 2 g of PVP was then added thereto and completely dissolved, giving a clear solution. 1 g of sodium lauryl sulfate was added to the solution, together with 5 ml distilled water, and completely dissolved to give a clear solution. The resultant reaction mixture was sprayed and then dried to produce a solid powder preparation using a procedure as will be described in Experimental Example 1, below.

### EXAMPLE 10

1 g of aceclofenac, 0.25 g of Tween 80, 0.25 g of oleic acid and 0.25 g of PEG 60 hydrogenated caster oil (Nikkol HCO-60) were dissolved in 20 ml of a mixture of acetone and ethanol in a volume ratio of 1:1, and 2 g of PVP was then added thereto and completely dissolved, giving a clear solution. 1 g of sodium lauryl sulfate was added to the solution, together with 5 ml distilled water, and completely dissolved to give a clear solution. The resultant reaction mixture was sprayed and then dried to produce a solid powder preparation using a procedure as will be described in Experimental Example 1, below.

### EXAMPLE 11

1 g of aceclofenac, 0.25 g of Tween 80, 0.25 g of oleic acid, and 0.25 g of glyceryl stearate/PEG 100 stearate (Arlacel 165) were dissolved in 20 ml of a mixture of acetone and ethanol in a volume ratio of 1:1, and 2 g of PVP was then added thereto and completely dissolved, giving a clear solution. 1 g of sodium lauryl sulfate was added to the solution, together with 5 ml distilled water, and completely dissolved to give a clear solution. The resultant reaction mixture was sprayed and then dried to produce a solid powder preparation using a procedure as will be described in Experimental Example 1, below.

### EXAMPLE 12

1 g of aceclofenac, 0.25 g of Tween 80, 0.25 g of oleic acid and 0.025 mg of butylated hydroxytoluene were dissolved in 20 ml of a mixture of acetone and ethanol in a volume ratio of 1:1, and 2 g of PVP was then added thereto and completely dissolved, giving a clear solution. 1 g of sodium lauryl sulfate was added to the solution, together with 5 ml distilled water, and completely dissolved to give a clear solution. The resultant reaction mixture was sprayed and then dried to produce a solid powder preparation using a procedure as will be described in Experimental Example 1, below.

### EXAMPLE 13

1 g of aceclofenac, 0.25 g of Tween 80, 0.25 g of oleic acid and 0.25 mg of butylated hydroxytoluene were dissolved in 20 ml of a mixture of acetone and ethanol in a volume ratio of 1:1, and 2 g of PVP was then added thereto and completely dissolved, giving a clear solution. 1 g of sodium lauryl sulfate was added to the solution, together with 5 ml distilled water, and completely dissolved to give a clear solution. The resultant reaction mixture was sprayed and then dried to produce a solid powder preparation using a procedure as will be described in Experimental Example 1, below.

### EXAMPLE 14

1 g of aceclofenac, 0.25 g of Tween 80, 0.25 g of oleic acid and 2.5 mg of butylated hydroxytoluene were dissolved in 20 ml of a mixture of acetone and ethanol in a volume ratio of 1:1, and 2 g of PVP was then added thereto and completely dissolved, giving a clear solution. 1 g of sodium lauryl sulfate was added to the solution, together with 5 ml distilled water, and completely dissolved to give a clear solution. The resultant reaction mixture was sprayed and then dried to produce a solid powder preparation using a procedure as will be described in Experimental Example 1, below.

### EXAMPLE 15

1 g of aceclofenac, 0.25 g of Tween 80, 0.25 g of medium-chain triglyceride (MCT) and 2.5 mg of butylated hydroxytoluene were dissolved in 20 ml of a mixture of acetone and ethanol in a volume ratio of 1:1, and 2 g of PVP was then added thereto and completely dissolved, giving a clear solution. 1 g of sodium lauryl sulfate was added to the solution, together with 5 ml distilled water, and completely dissolved to give a clear solution. The resultant reaction mixture was sprayed and then dried to produce a solid powder preparation using a procedure as will be described in Experimental Example 1, below.

### EXAMPLE 16

1 g of aceclofenac, 0.25 g of Tween 80, 0.25 g of oleic acid and 2.5 mg of butylated hydroxytoluene were dissolved in 20 ml of a mixture of acetone and ethanol in a volume ratio of 1:1, and 2 g of ethylcellulose was then added thereto and completely dissolved, giving a clear solution. 1 g of sodium lauryl sulfate was added to the solution, together with 5 ml distilled water, and completely dissolved to give a clear solution. The resultant reaction mixture was sprayed and then dried to produce a solid powder preparation using a procedure as will be described in Experimental Example 1, below.

### EXAMPLE 17

1 g of aceclofenac, 0.25 g of Tween 80, 0.25 g of oleic acid and 2.5 mg of butylated hydroxytoluene were dissolved in 20 ml of a mixture of acetone and ethanol in a volume ratio of 1:1, and 2 g of hydroxypropylmethylcellulose (HPMC) was then added thereto and completely dissolved, giving a clear solution. 1 g of sodium lauryl sulfate was added to the solution, together with 5 ml distilled water, and completely dissolved to give a clear solution. The resultant reaction mixture was sprayed and then dried to produce a solid powder preparation using a procedure as will be described in Experimental Example 1, below.

### EXAMPLE 18

1 g of aceclofenac, 0.25 g of Tween 80, 0.25 g of oleic acid and 2.5 mg of butylated hydroxytoluene were dissolved in 20 ml of a mixture of acetone and ethanol in a volume ratio of 1:1, and 2 g of methylcellulose was then added thereto and completely dissolved, giving a clear solution. 1 g of sodium lauryl sulfate was added to the solution, together with 5 ml distilled water, and completely dissolved to give a clear solution. The resultant reaction mixture was sprayed and then dried to produce a solid powder preparation using a procedure as will be described in Experimental Example 1, below.

### EXAMPLE 19

Colloidal silicon dioxide (Cab-O-Sil) 2% or magnesium stearate 2%, functioning as a lubricant, was homogeneously mixed with the solid powder prepared in Examples 1 to 18, and an amount corresponding to 70 mg of a drug, aceclofenac, was filled into an empty hard-gelatin capsule, producing solid capsules.

### EXAMPLE 20

Microcrystalline cellulose (Avicel) 10% as a disintegrant and magnesium stearate 2% as a lubricant were homogeneously mixed with the solid powder prepared in Examples 1 to 18, and an amount corresponding to 70 mg of the drug, 325 mg, was tableted on a rotary press machine (12 stations, Korea Hydraulic Machinery Co., Ltd, Korea), producing tablets.

### EXAMPLE 21

Tablets prepared in Example 20 were ground and then sieved using a 40-60 mesh to produce microgranules with a uniform size. An amount corresponding to 70 mg of the drug was filled into an empty hard-gelatin capsule, producing solid capsules.

### EXAMPLE 22

1 g of aceclofenac, 2 g of Tween 80, 2 g of oleic acid, 25 mg of butylated hydroxytoluene and 1 g of sodium lauryl sulfate were milled and well mixed, and 0.5 g of PVP was then added thereto and homogeneously mixed. A viscous semi-solid preparation was prepared according to the same method as will be described in Experimental Example 2, below.

### EXAMPLE 23

1 g of aceclofenac, 2 g of Tween 80, 2 g of oleic acid, 25 mg of butylated hydroxytoluene and 1 g of sodium lauryl sulfate were milled and well mixed, and 0.5 g of hydroxypropylmethylcellulose was then added thereto and homogeneously mixed. A viscous semi-solid preparation was prepared according to the same method as will be described in Experimental Example 2, below.

### EXAMPLE 24

1 g of aceclofenac, 2 g of Tween 80, 2 g of oleic acid, 25 mg of butylated hydroxytoluene and 1 g of sodium lauryl sulfate were milled and well mixed, and 0.5 g of sodium carboxymethylcellulose was then added thereto and homogeneously mixed. A viscous semi-solid preparation was prepared according to the same method as will be described in Experimental Example 2, below.

### EXAMPLE 25

1 g of aceclofenac, 2 g of Tween 80, 2 g of oleic acid, 25 mg of butylated hydroxytoluene and 1 g of sodium lauryl sulfate were milled and well mixed, and 0.25 g of glycerylmonostearate was then added thereto and homogeneously mixed. A viscous semi-solid preparation was prepared according to the same method as will be described in Experimental Example 2, below.

### EXAMPLE 26

1 g of aceclofenac, 2 g of Tween 80, 2 g of oleic acid, 25 mg of butylated hydroxytoluene and 1 g of sodium lauryl sulfate were milled and well mixed, and 0.25 g of carbamer was then added thereto and homogeneously mixed. A viscous semi-solid preparation was prepared according to the same method as will be described in Experimental Example 2, below.

### EXAMPLE 27

1 g of aceclofenac, 2 g of Tween 80, 2 g of oleic acid, 25 mg of butylated hydroxytoluene and 1 g of sodium lauryl sulfate were milled and well mixed, and 2.0 g of poloxamer melted at about 4° C. was then slowly added thereto and homogeneously mixed. A viscous semi-solid preparation was prepared according to the same method as will be described in Experimental Example 2, below.

### EXAMPLE 28

Viscous semi-solid preparations prepared in Examples 22 to 27 were filled into soft capsules in an amount corresponding to 70 mg of the drug, producing semi-solid capsules.

### EXAMPLE 29

1 g of aceclofenac, 0.25 g of Tween 80, 0.25 g of oleic acid, 1 g of sodium lauryl sulfate and 2 g of PVP were dissolved in 5 ml of a mixture of acetone and ethanol in a volume ratio of 1:1, along with 1.25 ml distilled water, heating at about 50° C. Using the mixture, solid powder was prepared according to the same method as will be described in Experimental Example 3, below.

### EXAMPLE 30

1 g of aceclofenac, 0.25 g of Tween 80, 0.25 g of oleic acid, 1 g of sodium lauryl sulfate, 2.5 mg of butylated hydroxytoluene and 2 g of PVP were dissolved in 5 ml of a mixture of acetone and ethanol in a volume ratio of 1:1, along with 1.25 ml distilled water, heating at about 50° C. Using the mixture, solid powder was prepared according to the same method as will be described in Experimental Example 3, below.

### EXAMPLE 31

Solid powder preparations prepared in Examples 29 and 30 were homogeneously mixed with 2% colloidal silicon dioxide (Cab-O-Sil) or magnesium stearate, functioning as a lubricant, and an amount corresponding to 70 mg of the drug was filled into an empty hard-gelatin capsule, producing solid capsules.

### EXAMPLE 32

Solid powder preparations prepared in Examples 29 and 30 were homogeneously mixed with 10% microcrystalline cellulose (Avicel) as a disintegrant and 2% magnesium stearate as a lubricant, and an amount containing 70 mg of the drug, 325 mg, was tableted on a rotary press machine (12 stations, Korea Hydraulic Machinery Co., Ltd, Korea), producing tablets.

### EXAMPLE 33

Tablets prepared in Example 32 were ground and then sieved using a 40-60 mesh to produce microgranules with a uniform size. An amount corresponding to 70 mg of the drug was filled into an empty hard-gelatin capsule, producing solid capsules.

### EXAMPLE 34

1 g of aceclofenac, 2 g of Tween 80, 2 g of oleic acid, 25 mg of butylated hydroxytoluene and 1 g of sodium lauryl sulfate were milled and well mixed, and 1 g of polyethylene glycol 6000 melted at about 60-80° C. under a reduced pressure and heating condition was then added thereto and homogeneously mixed, following by cooling to room temperature. A viscous semi-solid preparation was prepared according to the same method as will be described in Experimental Example 3, below.

### COMPARATIVE EXAMPLE 1

### Airtal as a Commercially Available Tablet

Airtal containing 100 mg of aceclofenac, which is commercially available in a tablet form, was used as a comparative sample.

### COMPARATIVE EXAMPLE 2

### Powdered Airtal as a Commercially Available Tablet

Airtal containing 100 mg of aceclofenac was finely ground and then preparaed to a solid powder preparation.

### COMPARATIVE EXAMPLE 3

### A Solid Powder Preparation Prepared by the Spray-Drying, Comprising a Drug and PVP

1 g of aceclofenac was dissolved in 25 ml of a mixture of acetone, ethanol and water in a volume ratio of 1:1:0.5, and 1 g of PVP was then added thereto and completely dissolved until being transparent. The resultant solution was spray-dried according to the same method as will be described in Experimental Example 1, below, producing a solid powder preparation.

### COMPARATIVE EXAMPLE 4

### A solid Powder Preparation Prepared by the Spray-Drying, Comprising a Drug and a Surfactant

1 g of aceclofenac was dissolved in 20 ml of a mixture of acetone and ethanol in a volume ratio of 1:1, and 0.5 g of sodium lauryl sulfate was added thereto, along with 5 ml distilled water, and completely dissolved until being transparent. The resultant solution was spray-dried according to the same method as will be described in Experimental Example 1, below, producing a solid powder preparation.

### COMPARATIVE EXAMPLE 5

### A Solid Powder Preparation Prepared by the Spray-Drying, Comprising a Drug and a Surfactant

1 g of aceclofenac was dissolved in 20 ml of a mixture of acetone and ethanol in a volume ratio of 1:1, and 1 g of sodium lauryl sulfate was added thereto, along with 5 ml distilled water, and completely dissolved until being transparent. The resultant solution was spray-dried according to the same method as will be described in Experimental Example 1, below, producing a solid powder preparation.

### COMPARATIVE EXAMPLE 6

### A Solid Powder Preparation Prepared by the Spray-Drying, Comprising a Drug and a Surfactant

1 g of aceclofenac was dissolved in 20 ml of a mixture of acetone and ethanol in a volume ratio of 1:1, and 2 g of sodium lauryl sulfate was added thereto, along with 5 ml distilled water, and completely dissolved until being transparent. The resultant solution was spray dried according to the same method as will be described in Experimental Example 1, below, producing a solid powder preparation.

### EXPERIMENTAL EXAMPLE 1

### Preparation of Solid Powder Preparation Containing Aceclofenac by the Spray-Drying

The mixture containing aceclofenac, oil, fatty acid and the surfactant, composed in the above Examples, was dissolved or dispersed in 25 ml of a distilled water, acetone, or a solvent mixture of acetone and ethanol. The mixture was sprayed, using a spray-drier (Me Hyun Engineering Co., Ltd, Korea) under the conditions of preheating time of about 10 min to 1 hr, spray-drying rate of about 5-20 ml/min, spraying temperature of 70-150° C., and rotary nozzle gun of 30-60 Hertz, or using a fluid-bed sprayer (Nero Aeromatic) under the conditions of transport rate of 2-8 ml/min and spray temperature of 40-90° C., producing a large quantity of solid powder. The solid powder prepared using the two sprayers does not show the difference in terms of formulation, density and dissolution rate, as will be demonstrated in Experimental Examples 4 and 5, below.

### EXPERIMENTAL EXAMPLE 2

### Preparation of Semi-Solid Preparation Containing Aceclofenac by a Mixing

The mixture containing aceclofenac, fatty acid, the surfactant and the additive (for example, antioxidant), composed in the Examples was milled and homogeneously mixed. The mixture was again homogenously mixed with a polymeric base (for example, PEG) melted under reduced pressure and heating, a polymeric base (for example, PVP, hydroxypropylmethylcellulose, sodium carboxymethylcellulose, glycerylmonostearate, and carbamer) capable of being dispersed in a mixed state, or a polymeric base (for example, poloxamer) melted at about 4° C., then formulated at room temperature to produce a viscous semi-solid preparation.

### EXPERIMENTAL EXAMPLE 3

### Preparation of Solid Powder Preparation Containing Aceclofenac by the Coprecipitation (Dissolve-Drying)

The mixture containing aceclofenac, oil, fatty acid, the surfactant and the polymeric base, composed in the above Example was dissolved or dispersed in 5 ml of acetone or a mixture of acetone and ethanol. After being heated and then supplemented with small amount of distilled water, the mixture was dried naturally or at about 50° C. The resultant powder was milled and sieved using a 60 mesh to obtain solid powder having a uniform size.

### EXPERIMENTAL EXAMPLE 4

### Measurement of Content of Acelcofenac Contained in the Pharmaceutical Preparation

The pharmaceutical preparation containing acelcofenac was completely dissolved in 500 ml of an ethanol solution containing phosphate buffer (pH 6.8) at a volume ratio of 50%, where a preparation further containing substances of low solubility was shaken for 10 min. The resultant solution was centrifuged at 15,000 rpm for 2 min, and then filtered with a 0.45 μm membrane filter. After properly diluting 1 ml of the filtered solution, sample was used in quantification of aceclofenac, using HPLC. In a analysis of aceclofenac, a column was C18 ODS column (4.6×150 mm, 5 μm), an absorbance was at a wave length of 282 nm, a mobile phase was a mixture of MeOH:0.02 M KH₂PO₄ (65:35), a flow rate was 1 ml/min. 20 μl of sample was injected. Quantification of aceclofenac was accomplished with width ratio of aceclofenac and internal standard (Ethyl paraben).

### EXPERIMENTAL EXAMPLE 5

### Measurement of Dissolution Rate of Acelcofenac Contained in the Pharmaceutical Preparation

Dissolution rate of acelcofenac contained in the pharmaceutical preparation was analyzed according to the dissolution test method disclosed in a guidebook “Korea Pharmacopeia (7^{th} revision)”. A NaCl—HCl buffer solution (pH 1.4±0.1) was used as artificial gastric juice, supplemented with Tween 80 in a volume ratio of 0.3% according to intended use. 0.02 M phosphate-buffered solution (pH 6.8±0.1) was used as an artificial intestinal juice. Dissolution was performed according to the paddle method at a stirring rate of 50 rpm and a dissolution temperature of 37±0.5° C., using 500 ml of dissolution solution. 0.5 ml samples were collected at 0, 2, 5, 10, 15, 30, 60, and 90 min, at which point the test solution was supplemented with an equivalent amount of dissolution solution. The collected sample was centrifuged at 15,000 rpm for 2 min and filtered using a 0.45 μm membrane filter. Quantification of aceclofenac was accomplished by using HPLC. In analyzing the dissolution rate of aceclofenac, column was C18 ODS column (4.6×150 mm, 5 μm), absorbance was a wave length of 282 nm, a mobile phase was a mixture of MeOH:0.02 M KH₂PO₄ (65:35), and a flow rate was 1 ml/min. 20 μl of sample was injected. Quantification of aceclofenac was accomplished with width ratio of aceclofenac and internal standard (Ethyl paraben) Dissolution rate (%) of aceclofenac contained in the solid powder preparations, prepared in example, was measured according to Experimental 5. Content of aceclofenac obtained in Experimental Example 4 was used as 100% and dissolution rate of aceclofenac contained in the solid powder preparations prepared in example was given as a percentage. Dissolution concentration (μg/ml) and dissolution rate (%) of aceclofenac contained in solid powder preparation in artificial gastric juice were measured, the results are given in Tables 1 to 3, below,

TABLE 1
Dissolution concentration (μg/ml) and dissolution rate (%)
of aceclofenac contained in the solid powder preparations in
the pure artificial gastric juice

| | Time (min) | | | | |
|---|---|---|---|---|---|
| preparation | 15 | 30 | 45 | 60 | 90 |
| Aceclofenac | 0.075 | 0.063 | 0.088 | 0.090 | 0.236 |
| | (0.04) | (0.03) | (0.05) | (0.05) | (0.12) |
| Example 1 | 21.497 | 20.903 | 20.203 | 21.469 | 11.430 |
| (spray-drying) | (9.43) | (9.16) | (6.86) | (9.45) | (5.00) |
| Example 2 | 65.435 | 45.719 | 37.644 | 32.186 | 29.847 |
| (spray-drying) | (36.81) | (25.72) | (21.18) | (18.11) | (16.79) |
| Example 3 | 17.069 | 9.746 | 6.627 | 7.269 | 3.659 |
| (spray-drying) | (9.87) | (5.64) | (3.83) | (4.20) | (2.12) |
| Example 4 | 80.337 | 54.328 | 39.141 | 25.268 | 21.510 |
| (spray-drying) | (40.17) | (27.16) | (19.57) | (12.63) | (10.76) |
| Comparative example | 3.388 | 1.500 | 1.610 | 1.319 | 1.181 |
| 1 (spray-drying) | (1.69) | (0.75) | (0.81) | (0.66) | (0.59) |
| Comparative example | 3.825 | 4.603 | 4.986 | 4.851 | 5.081 |
| 3 (spray-drying) | (1.91) | (2.30) | (2.49) | (2.43) | 92.54) |
| Comparative example | 42.549 | 25.947 | 18.619 | 14.047 | 12.754 |
| 4 (spray-drying) | (21.27) | (12.97) | (9.31) | (7.02) | (6.38) |
| Comparative example | 14.368 | 8.900 | 6.353 | 6.190 | 4.246 |
| 5 (spray-drying) | (7.93) | (4.92) | (3.51) | (3.42) | (2.34) |
| Comparative example | 16.377 | 14.602 | 9.765 | 8.252 | 5.198 |
| 6 (spray-drying) | (8.52) | (7.60) | (5.08) | (4.29) | (2.70) |

In the artificial gastric juice not containing 0.3% of Tween 80, the solid powder preparations prepared in Examples were found to have dissolution rates higher than that of aceclofenac powder, the solid powder prepared in Comparative Example 3 or the commercially available preparation, while all pharmaceutical preparations showed a sharp decrease in dissolution rate with the lapse of time.

TABLE 2
Dissolution concentration (μg/ml) and dissolution rate (%)
of aceclofenac contained in the solid powder preparations in
the artificial gastric juice containing 0.3% of Tween 80

| | Time (min) | | | | |
|---|---|---|---|---|---|
| Preparation | 15 | 30 | 60 | 90 | 120 |
| Example 4 | 201.84 | 204.25 | 203.44 | 203.55 | 204.81 |
| (spray-drying) | (100.90 | (102.10 | (101.77) | (101.7) | (102.4) |
| Example 5 | 154.75 | 156.89 | 158.78 | 157.85 | 156.71 |
| (spray-drying) | (77.3) | (78.4) | (79.3) | (78.93) | (78.3) |
| Example 6 | 124.84 | 136.01 | 135.90 | 131.51 | 135.80 |
| (spray-drying) | (62.4) | (68.0) | (67.9) | (65.7) | (67.9) |

The solid powder prepared in Example 4 showed a very high dissolution rate of nearly 100% with no appearance of deposits, indicating that the composition of a dissolution solution is important for evaluating dissolution rate. However, as the result of the solid powder preparation obtained from Example 5 and 6, as the concentration of the drug increases, dissolution rate of the aceclofenac decreases slightly

TABLE 3
Dissolution concentration (μg/ml) and dissolution rate (%)
of aceclofenac contained in the pharmaceutical preparations
for oral administration in pure the artificial gastric juice

| | Time (min) | | | | | | | |
|---|---|---|---|---|---|---|---|---|
| Preparation | 2 | 5 | 10 | 15 | 30 | 60 | 90 | 120 |
| Example | 48.1 | 63.8 | 52.1 | 51.8 | 52.1 | 49.9 | 41.6 | 36.5 |
| 7 (spray- | (24.0) | (31.9) | (26.0) | (25.9) | (26.0) | (24.9) | (20.8) | (18.2) |
| drying) | | | | | | | | |
| Example | 70.8 | 68.6 | 61.2 | 58.09 | 56.9 | 50.7 | 51.8 | 36.9 |
| 8 (spray- | (35.4) | (34.3) | (30.6) | (29.0) | (28.4) | (25.3) | (25.9) | (18.4) |
| drying) | | | | | | | | |
| Example 9 | 199.6 | 199.7 | 191.36 | 188.8 | 183.5 | 178.9 | 176.5 | 174.2 |
| Example 12-14 | (99.7) | (99.8) | (65.6) | (94.4) | (91.7) | (89.4) | (88.2) | (87.1) |
| (spray- | | | | | | | | |
| drying) | | | | | | | | |
| Example 10 | 175.0 | 183.2 | 192.70 | 186.6 | 182.3 | 159.7 | 174.4 | 165.4 |
| (spray- | (87.5) | (91.5) | (96.3) | (93.34) | (91.19) | (79.89) | (87.2) | (82.7) |
| drying) | | | | | | | | |
| Example 11 | 145.9 | 172.2 | 193.62 | 192.9 | 201.9 | 201.8 | 201.2 | 201.5 |
| (spray- | (72.9) | (86.0) | (96.8) | (96.4) | (100.9) | (100.9) | (100.6) | (100.8) |
| drying) | | | | | | | | |
| Example 16 | 88.3 | 117.3 | 121.7 | 126.6 | 128.1 | 131.7 | 136.6 | 136.6 |
| (spray- | (44.2) | (58.7) | (60.8) | (63.3) | (64.1) | (65.9) | (68.3) | (68.3) |
| drying) | | | | | | | | |
| Example 17 | 146.5 | 154.4 | 155.8 | 159.0 | 162.0 | 160.5 | 162.2 | 161.5 |
| (spray- | (73.3) | (77.7) | (77.9) | (79.5) | (81.0) | (80.1) | (81.1) | (80.7) |
| drying) | | | | | | | | |
| Example 18 | 133.7 | 136.5 | 136.6 | 137.7 | 142.6 | 145.7 | 143.7 | 143.1 |
| (spray- | (66.7) | (68.3) | (68.3) | (68.9) | (71.3) | (72.9) | (71.8) | (72.2) |
| drying) | | | | | | | | |
| Example 25 | 98.0 | 100.0 | 102.2 | 104.6 | 108.6 | 110.3 | 109.0 | 110.0 |
| (semi- | (49.0) | (50.0) | (61.1) | (52.3) | (54.3) | (55.2) | (54.5) | (55.0) |
| solid) | | | | | | | | |
| Example 27 | 108.8 | 109.0 | 110.0 | 112.5 | 114.0 | 115.0 | 116.2 | 116.0 |
| (semi- | (54.4) | (54.5) | (55.0) | (56.3) | (57.0) | (57.5) | (58.1) | (58.0) |
| solid) | | | | | | | | |
| Example 30 | 196.8 | 197.7 | 194.6 | 190.8 | 192.0 | 190.4 | 189.5 | 188.0 |
| (coprecipitation | (98.4) | (98.8) | (97.3) | (95.4) | (96.0) | (95.2) | (94.8) | (94.0) |

Even in the artificial gastric juice not containing 0.3% of Tween 80, the solid powder preparation prepared in Examples 9 to 14, which were prepared by adding the surfactant, oleic acid, fatty acid or fatty acid alcohol, etc. to the polymeric base and using the spray-drying, showed a very high dissolution rate of nearly 100%. Also, the antioxidant butylated hydroxytoluene, serving as a stabilizer, does not negatively affect the dissolution rate. On the other hand, the preparations prepared in Examples 16 to 18, which were prepared using PVP and other polymeric bases (ethylcellulose, hydroxypropylmethylcellulose, and methylcellulose) according to the spray-drying, showed lower dissolution rate than the preparations prepared in Examples 9, 12-14, but higher dissolution rates than the aceclofenac powder and the commercially available preparation.

On the other hand, the viscous semi-solid preparations prepared in Examples 25 and 27 using a variety of polymeric bases were found to have dissolution rate lower than the solid powder preparation prepared by the spray-drying or coprecipitation, whereas higher dissolution rates than the aceclofenac powder and the commercially available preparation, thus allowing various formulations of the pharmaceutical preparation.

In addition, the solid powder prepared in Example 30 according to the coprecipitation showed a dissolution rate much higher than the viscous semi-solid preparations, the commercially available preparation and aceclofenac powder, while having a dissolution rate similar to the solid powders prepared by the spray-drying.

### EXPERIMENTAL EXAMPLE 6

### Concentration of Aceclofenac Contained in Mouse Blood in Case of Aceclofenac Preparation and Commercially Available Preparation

After male white mice (Sprague-Dawley) having body weights of 250-310 g, purchased from Korea National Institute of Health, were adjusted to a new environment for about 1-2 weeks, it was used in experimental example. Mice were not fed for one day before the experiment and anesthetized with ether, and cannulation to its left femur artery was performed to inject a tube connected to a syringe containing 50 IU/ml heparin. When mice come out from the anesthesia after about 2 hours, a suspension of the solid powder preparation according to the present invention or the commercially available preparation was administered to mice using a sonde for oral administration in an amount of 20 mg aceclofenac per kg, and blood was then collected from left femur artery at 0, 15, 30, 45, 60, 90, 120, 180, and 240 min after administration. The collected blood was centrifuged at 3500 rpm for 10 min, and the blood plasma was isolated and stored at −20° C. until analysis. Concentration of aceclofenac in blood was determined by HPLC analysis. 300 μl of blood was put into a microtube, and 50 μl of a solution containing an internal standard substance and 600 μl of acetonitrile were then added to the tube. The mixture was vortexed for 2 min and then centrifuged at 15,000 rpm for 2 min. The supernant was isolated and 60 μl of the supernant was injected to HPLC. In analyzing the dissolution rate of aceclofenac, a column was C18 ODS column (4.6×150 mm, 5 μm), an absorbance was a wave length of 282 nm, a mobile phase was a mixture of MeOH:0.02 M KH₂PO₄ (65:35), and a flow rate was 1 ml/min, content of injected sample was 20 μl. Quantification of aceclofenac was accomplished with width ratio of aceclofenac and internal standard (Ethyl paraben). Plasma concentration of aceclofenac contained in the oral preparation according to the present invention and the commercially available preparation was measured, and the results are shown in Table 4, below.

TABLE 4
Plasma concentration (μg/ml) of aceclofenac in mice in case
of aceclofenac preparation and commercially available
preparation according to the time after oral administration

| | Time (min) | | | | | | |
|---|---|---|---|---|---|---|---|
| Preparation | 15 | 30 | 45 | 60 | 90 | 120 | 180 |
| Example 4 | 4.178 | 4.501 | 3.787 | 2.467 | 1.057 | 0.528 | 0.121 |
| (spray-drying) | | | | | | | |
| Example 9 | 8.231 | 3.978 | 2.920 | 1.715 | 0.869 | 0.664 | 0.405 |
| (spray-drying) | | | | | | | |
| Example 11 | 2.567 | 3.044 | 2.388 | 1.528 | 0.803 | 0.469 | 0.313 |
| (spray-drying) | | | | | | | |
| Example 22 | 3.671 | 4.211 | 3.242 | 1.528 | 0.752 | 0.475 | 0.148 |
| (semi-solid) | | | | | | | |
| Example 30 | 8.523 | 4.201 | 2.910 | 1.567 | 0.656 | 0.469 | 0.101 |
| (coprecipitation | | | | | | | |
| Comparative | 5.973 | 3.268 | 1.815 | 1.120 | 0.448 | 0.242 | 0 |
| example 2 | | | | | | | |
| (commercially | | | | | | | |
| available | | | | | | | |

When the oral preparation prepared in Example 9 according to the spray-drying or in Example 30 according to coprecipitation respectively, was administered to mice, it was found that concentration of aceclofenac in mice blood is much higher than that of the commercially available preparation or the other oral preparations of the present invention. However, despite having excellent dissolution ability, the oral preparation prepared in Example 11 showed lower bioavailabilty than the preparation prepared in Example 9. On the other hand, in the case of the viscous semi-solid preparation prepared in Example 22, it was observed that its bioavailabilty was slightly lower than that of the preparation prepared in Example 9, while much higher than that of the aceclofenac powder.

### EXPERIMENTAL EXAMPLE 7

### Plasma Concentration of Aceclofenac Contained in Human in Case of Aceclofenac Preparation and Commercially Available Preparation

The pharmaceutical preparation containing aceclofenac was administered to 6 healthy fasted human adult males aged 20-40, together with 300 ml water. 0, 0.5, 1, 1.5, 2, 3, 5, 8, and 12 hr after the administration, 10 ml blood was collected from their arms using catheters, and put into vacutainer tubes, followed by addition of heparin to prevent blood clotting. The volunteers were allowed to take small drinks after 3 hr and Gimbap, rice roll with seaweed that is kind of Korean food, after 10 hr. 8 hr and 10 hr after the administration, drinks and a bowl of boiled rice mixed with some vegetables were supplied to them, respectively. During the experiment, drinking of alcoholic beverages or caffeine was prohibited, and activity was limited to reading and sleeping. The collected blood was centrifuged at 3500 rpm for 10 min, and the isolated blood plasma using iron-free tubes was stored at −20° C. until analysis. To determine concentration of aceclofenac in human blood, HPLC was carried out as follows. 300 μl of blood was put into a microtube, and 50 μl of an internal standard substance and 600 μl of acetonitrile were then added to the tube, followed by vortexing for 2 min and then centrifuging at 15,000 rpm for 2 min. 60 μl of the supernatent was applied to HPLC. In analyzing the dissolution rate of aceclofenac, column was C18 ODS column (4.6×150 mm, 5 μm), absorbance was a wave length of 282 nm, a mobile phase was a mixture of MeOH:0.02 M KH₂PO₄ (65:35), and a flow rate was 1 ml/min, content of injected sample was 20 μl. Quantification of aceclofenac was accomplished with width ratio of aceclofenac and internal standard (Ethyl paraben).

After the capsule preparation containing 70 mg of aceclofenac and the commercially available preparation containing 100 mg of aceclofenac were orally administered, plasma concentration of aceclofenac in human was investigated according to time, and the results are shown in Table 5, below.

TABLE 5
Plasma concentration (μg/ml) of aceclofenac in human
according to the time after the capsule preparation
containing 70 mg of aceclofenac and the commercially
available preparation containing 100 mg of aceclofenac
were orally administered

| | Time (min) | | | | | | | |
|---|---|---|---|---|---|---|---|---|
| preparation | 30 | 60 | 90 | 120 | 180 | 300 | 480 | 720 |
| Example 9 | 1.387 | 8.003 | 6.938 | 4.989 | 1.985 | 0.596 | 0.297 | 0.179 |
| Comparative | 2.098 | 4.858 | 7.991 | 5.746 | 2.231 | 0.949 | 0.426 | 0.328 |
| example 1 | | | | | | | | |
| (Airtal) | | | | | | | | |

Pharmacokinetic parameters obtained from concentration of aceclofenac are given in Table 6, below. A biological equivalence was observed in a range of ±20%, in case of the capsule preparation containing 70 mg of aceclofenac and the commercially available one containing 100 mg of aceclofenac.

TABLE 6
Comparison between pharmacokinetic parameters

| | | | AUC (μg · |
|---|---|---|---|
| | Cₘₐₓ (μg/ml) | Tₘₐₓ (hour) | min/ml) |
| Example 9 | 9.05 ± 1.74 | 1.25 ± 0.42 | 17.77 ± 4.05 |
| Comparative | 9.10 ± 1.64 | 1.21 ± 0.46 | 18.85 ± 4.31 |
| example 1 | | | |
| (Airtal) | | | |

### EXPERIMENTAL EXAMPLE 8

### Test for Stability of the Capsule Preparation Containing Aceclofenac

The capsule prepared in Example 14, which contains 70 mg of aceclofenac, was put into a plastic bottle along with a drying agent, and then covered with a cap, without other auxiliary apparatuses. The bottle was left at 40° C. under 75% humidity. To estimate stability of aceclofenac, on the starting point, and after 1, 4, and 6 months, contents of aceclofenac in the capsule and dissolution rates were investigated according to the same method as in Examples 4 and 5, respectively.

Content of aceclofenac in the capsule containing 70 mg of aceclofenac when being prepared in Example 14 was investigated, and the result is given Table 7, below. As shown in Table 7, the capsule preparation was found to have excellent stability.

TABLE 7
Change in content of aceclofenac in the capsule
preparation according to time

Time (day)

| | 0 day | 1 month | 4 month | 6 month |
|---|---|---|---|---|
| Content (%) | 100.6 | 100.5 | 100.8 | 98.4 |

After the storage in the plastic bottle, dissolution rate of aceclofenac contained in the capsule preparation prepared in example 14 was analyzed, and the results are given in Table 8, below.

TABLE 8
Change (%) in dissolution rate of aceclofenac in the capsule
preparation according to time

Storage (day)

| Time (min) | 0 day | 1 month | 4 month | 6 month |
|---|---|---|---|---|
| 0 | 0 | 0 | 0 | 0 |
| 2 | 0 | 0 | 0 | 0 |
| 5 | 0.32 | 0.55 | 0.17 | 0.35 |
| 10 | 1.39 | 6.05 | 1.44 | 3.92 |
| 15 | 10.13 | 20.47 | 3.56 | 16.41 |
| 30 | 73.38 | 84.37 | 62.46 | 76.41 |
| 60 | 82.23 | 85.96 | 84.33 | 81.45 |
| 90 | 78.16 | 75.10 | 79.87 | 81.30 |
| 120 | 69.74 | 74.09 | 72.66 | 74.77 |

As shown in Table 8, no large change in dissolution rate of aceclofenac in the capsule preparation was found according to the time after storage at 40° C. under 75% humidity, demonstrating that the aceclofenac-containing solid capsule prepared in Example 14 is highly stable. Slightly changed dissolution pattern at early stages was originated from the different disruption times of capsules themselves, not influence of powdered ingredients. Since aceclofenac level in blood is similar to that of the commercially available preparation, the pharmaceutical preparation for oral administration according to the present invention can be used instead of the conventional preparations containing aceclofenac.

### INDUSTRIAL APPLICABILITY

As described hereinbefore, the oral preparation according to the present invention has excellent solubility in gastrointestinal tract, thereby improving dissolution rate and thus bioavailabilty, as well as rapid dispersion and dissolution properties in gastrointestinal tract. In addition, when being orally administered in an amount much smaller than the conventional preparations, the oral preparation according to the present invention is therapeutically effective, thus minimizing gastrointestinal disorders.

What is claimed is:

1. (deleted)

2. A pharmaceutical preparation for oral administration, comprising aceclofenac, polyvinylpyrrolidone (PVP) as a polymeric base and a surfactant.

3. The pharmaceutical preparation according to claim 2, wherein the preparation is formulated into solid powder, compressed particles, granules or tablets, capsules, or semisolid form.

4. The pharmaceutical preparation according to claim 2, wherein the preparation is prepared by spray-drying or dissolve-drying (coprecipitating) a solution containing aceclofenac, polyvinylpyrrolidone (PVP) as a polymeric base and a surfactant.

5. (deleted)

6. (deleted)

7. The pharmaceutical preparation according to claim 2, wherein the surfactant is selected from the group consisting of sodium lauryl sulfate and its derivatives, poloxamer and its derivatives, labrafil, labrafac, polysorbates, sorbitan esters, cremophor, medium chain triacylglyceride (MCT), PEG-60 hydrogenated castor oil, PEG-40 hydrogenated castor oil, sodium lauryl glutamate, disodium cocoamphodiacetate, and mixtures thereof.

8. The pharmaceutical preparation according to claim 7, wherein the surfactant is selected from the group consisting of sodium lauryl sulfate and its derivatives, polysorbates, sorbitan esters, and mixtures thereof.

9. The pharmaceutical preparation according to claim 7, wherein the surfactant is selected from the group consisting of sodium lauryl sulfate, Tween 20, Tween 40, Tween 60, Tween 80, and mixtures thereof.

10. The pharmaceutical preparation according to claim 2, further comprising one or more selected from the group consisting of fatty acid or fatty acid alcohol, oil, an antioxidant.

11. The pharmaceutical preparation according to claim 10, wherein the fatty acid or fatty acid alcohol is selected from the group consisting of oleic acid, stearyl alcohol, myristic acid, linoleic acid, lauric acid, capric acid, caprylic acid, caproic acid, and mixtures thereof.

12. The pharmaceutical preparation according to claim 11, wherein the fatty-acid or fatty acid alcohol is oleic acid.

13. (deleted)

14. The pharmaceutical preparation according to claim 10, wherein the antioxidant is selected from the group consisting of butylated hydroxytoluene, sodium bisulfite, α-tocorpherol, vitamin C, β-carotin, ascobylpamitate, tocopherol acetate, fumaric acid, nalic acid, butylated hydroxyanisole, propyl gallate, and sodium ascorbate.

15. (deleted)

16. (deleted).

17. The pharmaceutical preparation according to claim 2, wherein the composition comprises aceclofenac in an amount of 56 to 84 parts by weight, polyvinylpyrrolidone (PVP) as a polymeric base in an amount of 112 to 168 parts by weight, and the surfactant in an amount of 56 to 168 parts by weight.

18. The pharmaceutical preparation according to claim 2, wherein the composition comprises aceclofenac in an amount of 56 to 84 parts by weight, polyvinylpyrrolidone (PVP) as a polymeric base in an amount of 112 to 168 parts by weight, sodium lauryl sulfate in an amount of 56 to 84 parts by weight, Tween 80 in an amount of 15 to 20 parts by weight, fatty acid or fatty acid alcohol in an amount of 15 to 20 parts by weight, and the antioxidant in an amount of 0.15 to 0.20 parts by weight.

19. The pharmaceutical preparation according to claim 2, wherein the composition comprises 30 to 150 mg of aceclofenac.

20. (deleted)

21. The pharmaceutical preparation according to claim 19, wherein the composition is formulated into capsules containing 40 to 70 mg of aceclofenac.

22. The pharmaceutical preparation according to claim 2, wherein the composition is formulated into a pharmaceutical dosage form by dissolving aceclofenac, polyvinylpyrrolidone (PVP) as a polymeric base, and an the surfactant in a hydrophilic solvent or a mixture of the hydrophilic solvent and water, and then drying the solution.

23. The pharmaceutical preparation according to claim 22, wherein the hydrophilic solvent is selected from the group consisting of acetone, ethanol, and mixtures thereof.

24. The pharmaceutical preparation according to claim 2, wherein the composition is formulated into a pharmaceutical dosage form by dissolving aceclofenac, polyvinylpyrrolidone (PVP) as a polymeric base and the surfactant in a hydrophilic solvent or a solution of the hydrophilic solvent mixed with water, drying the solution and grinding, and formulating into compressed particles, granules, capsules or tablets.

25. (deleted)

26. A method of preparing a pharmaceutical preparation according to claim 2, comprising the steps of dissolving a mixture containing aceclofenac, polyvinylpyrrolidone (PVP) as a polymeric base and a surfactant in a hydrophilic solvent or a solution of the hydrophilic solvent mixed with water, and obtaining a solid powder preparation by drying the solution.

27. (deleted)

28. (deleted)

29. (deleted)

30. A method of preparing a semi-solid preparation for oral administration according to claim 2, comprising the steps of milling and well mixing a mixture of aceclofenac and a surfactant, adding polyvinylpyrrolidone (PVP) as a polymeric base thereto, and formulating into a viscous form.

31. The method according to claim 30, wherein the mixture further comprises fatty acid or fatty acid alcohol, and an antioxidant.

32. The method according to claim 31, wherein the mixture contains aceclofenac in an amount of 56 to 84 parts by weight, polyvinylpyrrolidone (PVP) as a polymeric base in an amount of 10 to 168 parts by weight, sodium lauryl sulfate in an amount of 56 to 84 parts by weight, Tween 80 in an amount of 50 to 160 parts by weight, fatty acid or fatty acid alcohol in an amount of 50 to 160 parts by weight, and the antioxidant in an amount of 1.5 to 2.0 parts by weight.
